SEQUENCE LISTING (6 sequences)

SEQ ID NO: 1 - protein, 4 aa, Artificial Sequence
  Description of Artificial Sequence Synthetic peptide
KYKK

SEQ ID NO: 2 - protein, 9 aa, Artificial Sequence
  Description of Artificial Sequence Synthetic peptide
FQWAVGHXL

SEQ ID NO: 3 - protein, 10 aa, Artificial Sequence
  Description of Artificial Sequence Synthetic peptide
QWVWAVGHLM

SEQ ID NO: 4 - protein, 4 aa, Artificial Sequence
  Description of Artificial Sequence Synthetic peptide
AKYK

SEQ ID NO: 5 - protein, 11 aa, Artificial Sequence
  Description of Artificial Sequence Synthetic peptide
AAFQWAVGHXL

SEQ ID NO: 6 - protein, 11 aa, Artificial Sequence
  Description of Artificial Sequence Synthetic peptide
AAFQWAVGHXL

(19) United States
(12) Patent Application Publication
(10) Pub. No.: US 2015/0217006 A1
(43) Pub. Date: Aug. 6, 2015

(54) AL-F-18-LABELED, AL-F-19-LABELED AND GA-68-LABELED GASTRIN-RELEASING PEPTIDE RECEPTOR (GRPR)-ANTAGONISTS FOR IMAGING OF PROSTATE CANCER

(71) Applicant: Immunomedics, Inc., Morris Plains, NJ (US)

(72) Inventors: William J. McBride, Boonton, NJ (US); Kristell L.S. Chatalic, Rotterdam (NL); Marion Hendriks-de Jong, Vlaardingen (NL); Otto C. Boerman, Malden (NL); David M. Goldenberg, Mendham, NJ (US)

(21) Appl. No.: 14/599,816

(22) Filed: Jan. 19, 2015

Related U.S. Application Data
(60) Provisional application No. 61/936,478, filed on Feb. 6, 2014.

(51) Int. Cl. A61K 51/08; A61K 49/14; A61K 49/00

(52) U.S. Cl. CPC A61K 51/088; A61K 49/0056; A61K 49/14

(57) ABSTRACT

The present application discloses compositions and methods of synthesis and use of ¹⁸F-, ¹⁹F- or ⁶⁸Ga-labeled molecules of use in PET, SPECT and/or MRI imaging of prostate cancer. Preferably, the ¹⁸F, ¹⁹F or ⁶⁸Ga is attached to a chelator moiety on a prostate cancer targeting molecule, more preferably a bombesin analog, more preferably a GRPR antagonist, most preferably JMV5132 or JMV4168. The ¹⁸F or ¹⁹F may form a complex with a group IIIA metal to promote binding to the chelators. The labeled molecules may be used to detect, diagnose and/or image prostate cancer, including metastatic prostate cancer, in vivo.

### RELATED APPLICATIONS

This application claims the benefit under 35 U.S.C. 119(e) of Provisional U.S. Patent Application Ser. No. 61/936,478, filed Feb. 6, 2014, the priority application incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Jan. 14, 2015, is named IMM333US1_SL.txt and is 2,713 bytes in size.

1. Field

The present invention concerns methods of labeling targeting peptides with ¹⁸F, ¹⁹F or ⁶⁸Ga that are of use, for example, in PET, SPECT or MRI in vivo imaging. Preferably, the ¹⁸F or ¹⁹F is attached as a complex with aluminum or another metal, such as a Group IIIA metal, via a chelating moiety, which may be covalently linked to a targeting peptide. ⁶⁸Ga may be directly attached to a chelating moiety without forming any complex. The chelating moiety may be attached to a protein or peptide either before or after binding of the chelating moiety to the metal-¹⁸F, metal-¹⁹F or ⁶⁸Ga. Although labeling may occur at an elevated temperature, such as 70° C., 80° C., 90° C., 95° C., 100° C., 105° C., 110° C., or any temperature in between, preferably labeling of heat sensitive molecules may occur at a lower temperature, such as room temperature. Most preferably, the metal-¹⁸F or metal-¹⁹F complex or ⁶⁸Ga is attached to the chelating moiety at elevated temperature, and the chelating moiety is than attached to a heat sensitive molecule at room temperature.

In certain embodiments, the labeled molecule may be used for targeting a diseased cell, tissue or organ to be imaged or detected, such as a tumor. Exemplary targeting molecules include, but are not limited to, an antibody, antigen-binding antibody fragment, bispecific antibody, affibody, diabody, minibody, scFvs, aptamer, avimer, targeting peptide, somatostatin, bombesin, bombesin analog, octreotide, RGD peptide, folate, folate analog or any other molecule known to bind to a disease-associated target. In preferred embodiments, the labeled molecule is a bombesin analog. Most preferably, the labeled molecule is a GRPR antagonist. Such molecules are of use for detection and/or imaging of GRPR⁺ cancer, for example prostate cancer.

Using the techniques described herein, Al¹⁸F-labeled, Al¹⁹F-labeled or ⁶⁸Ga-labeled molecules of high specific activity may be prepared in 30 minutes or less and are suitable for use in imaging techniques without the need for HPLC purification of the labeled molecule. Labeling may occur in a saline medium suitable for direct use in vivo. In alternative embodiments an organic solvent may be added to improve the labeling efficiency. The labeled molecules are stable under physiological conditions, although for certain purposes, such as kit formulations, a stabilizing agent such as ascorbic acid, trehalose, sorbitol or mannitol may be added. In other alternative embodiments, a chelating moiety may be preloaded with aluminum and lyophilized for storage, prior to labeling with ¹⁸F.

2. Background

Positron Emission Tomography (PET) has become one of the most prominent functional imaging modalities in diagnostic medicine, with very high sensitivity (fmol), high resolution (4-10 mm) and tissue accretion that can be adequately quantitated (Volkow et al., 1988, Am. J. Physiol. Imaging 3:142). Although [¹⁸F]2-deoxy-2-fluoro-D-glucose ([¹⁸F]FDG) is the most widely used PET imaging agent in oncology (Fletcher et al., 2008, J. Nucl. Med. 49:480), there is a keen interest in developing other labeled compounds for functional imaging to complement and augment anatomic imaging methods (Torigian et al., 2007, CA Cancer J. Clin. 57:206), especially with the hybrid PET/computed tomography systems currently in use. Thus, there is a need to have facile methods of conjugating positron emitting radionuclides to various molecules of biological and medical interest.

Peptides or other targeting molecules can be labeled with the positron emitters ¹⁸F, ⁶⁴Cu, ¹¹C, ⁶⁶Ga, ⁶⁸Ga, ⁷⁶Br, ^{94m}Tc, ⁸⁶Y, and ¹²⁴I. A low ejection energy for a PET isotope is desirable to minimize the distance that the positron travels from the target site before it generates the two 511 keV gamma rays that are imaged by the PET camera. Many isotopes that emit positrons also have other emissions such as gamma rays, alpha particles or beta particles in their decay chain. It is desirable to have a PET isotope that is a pure positron emitter so that any dosimetry problems will be minimized. The half-life of the isotope is also important, since the half-life must be long enough to attach the isotope to a targeting molecule, analyze the product, inject it into the patient, and allow the product to localize, clear from non-target tissues and then image. ¹⁸F and ⁶⁸Ga are two of the most widely used PET emitting isotopes because of their appropriate half-lifes and favorable positron emission characteristics.

Conventionally, ¹⁸F is attached to compounds by binding it to a carbon atom (Miller et al., 2008, Angew Chem Int Ed 47:8998-9033), but attachments to silicon (Shirrmacher et al., 2007, Bioconj Chem 18:2085-89; Hohne et al., 2008, Bioconj Chem 19:1871-79) and boron (Ting et al., 2008, Fluorine Chem 129:349-58) have also been reported. Binding to carbon usually involves multistep syntheses, including multiple purification steps, which is problematic for an isotope with a 110-min half-life, and typically results in poor radiochemical yields. Current methods for ¹⁸F-labeling of peptides typically involve the labeling of a reagent at low specific activity, HPLC purification of the reagent and then conjugation to the peptide of interest. The conjugate is often repurified after conjugation to obtain the desired specific activity of labeled peptide.

An example is the labeling method of Poethko et al. (J. Nucl. Med. 2004; 45: 892-902) in which 4-[¹⁸F]fluorobenzaldehyde is first synthesized and purified (Wilson et al, J. Labeled Compounds and Radiopharm. 1990;XXVIII: 1189-1199) and then conjugated to the peptide. The peptide conjugate is then purified by HPLC to remove excess peptide that was used to drive the conjugation to completion. Other examples include labeling with succinimidyl [¹⁸F]fluorobenzoate (SFB) (e.g., Vaidyanathan et al., 1992, Int. J. Rad. Appl. Instrum. B 19:275), other acyl compounds (Tada et al., 1989, Labeled Compd. Radiopharm.XXVII:1317; Wester et al., 1996, Nucl. Med. Biol. 23:365; Guhlke et al., 1994, Nucl. Med. Biol 21:819), or click chemistry adducts (Li et al., 2007, Bioconj Chem. 18:1987). The total synthesis and formulation time for these methods ranges between 1-3 hours, with most of the time dedicated to the HPLC purification of the labeled peptides to obtain the specific activity required for in vivo targeting. With a 2 hr half-life, all of the manipulations that are needed to attach the ¹⁸F to the peptide are a significant burden. These methods are also tedious to perform and require the use of equipment designed specifically to produce the labeled product and/or the efforts of specialized professional chemists. They are also not conducive to kit formulations that could routinely be used in a clinical setting.

A need exists for a rapid, simple method of ¹⁸F, ¹⁹F or ⁶⁸Ga labeling of targeting moieties, such as proteins or peptides, preferably at high radiochemical yield, which results in targeting constructs of suitable specific activity and in vivo stability for detection and/or imaging, while minimizing the requirements for specialized equipment or highly trained personnel and reducing operator exposure to high levels of radiation. An additional need exists for methods of efficiently labeling temperature sensitive molecules.

### SUMMARY

In various embodiments, the present invention concerns compositions and methods relating to ¹⁸F-labeled, ¹⁹F-labeled or ⁶⁸Ga-labeled molecules of use for PET imaging. In an exemplary approach, the ¹⁸F or ¹⁹F is bound to a metal and the ¹⁸F-metal or ¹⁹F-metal complex, or ⁶⁸Ga, is attached to a chelating moiety on a targeting peptide, such as a GRPR antagonist. As described below, the metals of group IIIA (aluminum, gallium, indium, and thallium) are suitable for ¹⁸F or ¹⁹F binding, although aluminum is preferred. Lutetium may also be of use. The chelating moiety may be selected from NOTA, NODA, NETA, DOTA, DTPA and other chelating groups discussed in more detail below. In other embodiments, one may attach an ¹⁸F-metal, ¹⁹F-metal or ⁶⁸Ga to a chelating moiety first and then attach the labeled chelating moiety to a molecule, such as a temperature sensitive molecule. In this way, the ¹⁸F-metal, ¹⁹F-metal or ⁶⁸Ga may be attached to a chelating moiety at a higher temperature, such as between 90° to 110° C., more preferably between 95° to 105° C., and the ¹⁸F-metal, ¹⁹F-metal or ⁶⁸Ga-labeled chelating moiety may be attached to a temperature sensitive molecule at a lower temperature, such as at room temperature. In preferred embodiments, the labeling method uses a biofunctional chelator that forms a physiologically stable complex with metal-¹⁸F, metal-¹⁹F or ⁶⁸Ga, which contains reactive groups that can bind to proteins, peptides or other targeting molecules at, e.g., room temperature. More preferably, labeling can be accomplished in 10 to 15 minutes in aqueous medium, with a total synthesis time of about 30 minutes.

Exemplary targeting peptides described in the Examples below, of use for delivery of ¹⁸F, ¹⁸F or ⁶⁸Ga, include but are not limited to JMV594, JMV4168 and JMV5132. However, other bombesin analogs, more particularly other GRPR antagonists, may be labeled and used for detection, diagnosis and/or imaging, using the disclosed methods and compositions.

The chelating moieties of use are also not limited to the exemplary embodiments shown below. Successful labeling with Al-¹⁸F, Al-¹⁹F and/or ⁶⁸Ga has been demonstrated with chelating moieties such as DTPA, NOTA, benzyl-NOTA, alkyl or aryl derivatives of NOTA, NODA, NODA-GA, C-NETA, succinyl-C-NETA and bis-t-butyl-NODA. In a preferred embodiment, a chelating moiety based on NODA-propyl amine (e.g., (tBu)₂NODA-propyl amine) may be derivatized to form a reactive thiol, maleimide, azide, alkyne or aminooxy group, which may then be conjugated to a targeting molecule at a reduced temperature via azide-alkyne coupling, thioether, amide, dithiocarbamate, thiocarbamate, oxime or thiourea formation.

In preferred embodiments, molecules that bind directly to receptors, such as somatostatin, octreotide, bombesin, a bombesin analog, folate or a folate analog, an RGD peptide or other known receptor ligands may be labeled and used for imaging. Receptor targeting agents may include, for example, TA138, a non-peptide antagonist for the integrin αᵥβ₃ receptor (Liu et al., 2003, Bioconj. Chem. 14:1052-56). Other methods of receptor targeting imaging using metal chelates are known in the art and may be utilized in the practice of the claimed methods (see, e.g., Andre et al., 2002, J. Inorg. Biochem. 88:1-6; Pearson et al., 1996, J. Med., Chem. 39:1361-71).

In more preferred embodiments, metal-¹⁸F, metal-¹⁹F or ⁶⁸Ga may be attached to a bombesin (BBN) analog that is a GRPR antagonist, such as JMV594, JMV4168 or JMV5132 for labeling and distribution studies of the gastrin-releasing peptide receptor (GRPR), which is overexpressed in human cancers such as prostate cancer. PET or SPECT imaging of labeled BBN analogs may also be used for detection or diagnosis of tumors that express GRPR. The type of diseases or conditions that may be imaged is limited only by the availability of a suitable delivery molecule for targeting a cell or tissue associated with the disease or condition. Many such delivery molecules are known. For example, any protein or peptide that binds to a diseased tissue or target, such as cancer, may be labeled with ¹⁸F, ¹⁹F or or ⁶⁸Ga by the disclosed methods and used for detection and/or imaging. In certain embodiments, such proteins or peptides may include, but are not limited to, antibodies or antibody fragments that bind to tumor-associated antigens (TAAs). Any known TAA-binding antibody or fragment may be labeled with ¹⁸F, ¹⁹F or ⁶⁸Ga by the described methods and used for imaging and/or detection of tumors, for example by PET, SPECT, MRI or other known techniques.

Certain alternative embodiments involve the use of “click” chemistry for attachment of ¹⁸F-, ¹⁹F- or ⁶⁸Ga-labeled moieties to targeting molecules. Preferably, the click chemistry involves the reaction of a targeting molecule such as an antibody or antigen-binding antibody fragment, comprising a functional group such as an alkyne, nitrone or an azide group, with a ¹⁸F-, ¹⁸F- or ⁶⁸Ga-labeled moiety comprising the corresponding reactive moiety such as an azide, alkyne or nitrone. Where the targeting molecule comprises an alkyne, the chelating moiety or carrier will comprise an azide, a nitrone or similar reactive moiety. The click chemistry reaction may occur in vitro to form a highly stable, ¹⁸F-, ¹⁹F- or ⁶⁸Ga-labeled targeting molecule that is then administered to a subject.

In other alternative embodiments, a prosthetic group, such as a NODA-maleimide moiety, may be labeled with ¹⁸F-metal, ¹⁹F-metal or ⁶⁸Ga and then conjugated to a targeting molecule, for example by a maleimide-sulfhydryl reaction. Exemplary NODA-maleimide moieties include, but are not limited to, NODA-MPAEM, NODA-PM, NODA-PAEM, NODA-BAEM, NODA-BM, NODA-MPM, and NODA-MBEM.

### BRIEF DESCRIPTION OF THE DRAWINGS

The following Figures are included to illustrate particular embodiments of the invention and are not meant to be limiting as to the scope of the claimed subject matter.

The following Figures are included to illustrate particular embodiments of the invention and are not meant to be limiting as to the scope of the claimed subject matter.

FIG. 1A. Chemical structure of DOTA-βAla-βAla-[D-Phe⁶,Sta¹³,Leu¹⁴]bombesin[6-14] (JMV4168 (SEQ ID NO: 6)).

FIG. 1B. Chemical structure of NODA-MPAA-βAla-βAla-[D-Phe⁶,Sta¹³,Leu¹⁴]bombesin[6-14] (JMV5132 (SEQ ID NO: 5)).

FIG. 2. Competition binding curves. PC-3 frozen sections were incubated in the presence of 5×10⁻¹⁰ M [¹²⁵I-Tyr⁴]BBN and increasing amounts of JMV4168, JMV5132, ^{nat}Ga-JMV4168 or ^{nat}Ga-JMV5132.

FIG. 3A. PET/CT images of mice bearing subcutaneous PC-3 xenografts on the right shoulder (arrow) injected with ⁶⁸Ga-JMV4168 (1), ⁶⁸Ga-JMV5132 (2) or Al¹⁸F-JMV5132 (3) at 1 h p.i.

FIG. 3B. PET/CT images of mice bearing subcutaneous PC-3 xenografts on the right shoulder (arrow) injected with ⁶⁸Ga-JMV4168 (1), ⁶⁸Ga-JMV5132 (2) or Al¹⁸F-JMV5132 (3) at 2 h p.i.

FIG. 3C. PET/CT images of mice bearing subcutaneous PC-3 xenografts on the right shoulder (arrow) injected with ⁶⁸Ga-JMV4168 (1), ⁶⁸Ga-JMV5132 (2) or Al¹⁸F-JMV5132 (3) at 2 h p.i. with co-injection of excess unlabeled peptide.

FIG. 4A. Biodistribution of⁶⁸Ga-JMV4168, ⁶⁸Ga-JMV5132 and Al¹⁸F-JMV5132 in mice bearing PC-3 xenografts at 1 h p.i. Int=intestines.

FIG. 4B. Biodistribution of ⁶⁸Ga-JMV4168, ⁶⁸Ga-JMV5132 and Al¹⁸F-JMV5132 in mice bearing PC-3 xenografts at 2 h p.i.

FIG. 4C. Biodistribution of⁶⁸Ga-JMV4168, ⁶⁸Ga-JMV5132 and Al¹⁸F-JMV5132 in mice bearing PC-3 xenografts at 2 h p.i. with co-injection of excess unlabeled peptide.

FIG. 4D. Biodistribution of⁶⁸Ga-JMV4168, ⁶⁸Ga-JMV5132 and Al¹⁸F-JMV5132 in mice bearing PC-3 xenografts, tumor-to-organ ratios at 2 h p.i.

### DETAILED DESCRIPTION

The following definitions are provided to facilitate understanding of the disclosure herein. Terms that are not explicitly defined are used according to their plain and ordinary meaning

As used herein, “a” or “an” may mean one or more than one of an item.

As used herein, the terms “and” and “or” may be used to mean either the conjunctive or disjunctive. That is, both terms should be understood as equivalent to “and/or” unless otherwise stated.

As used herein, “about” means within plus or minus ten percent of a number. For example, “about 100” would refer to any number between 90 and 110.

A “therapeutic agent” is an atom, molecule, or compound that is useful in the treatment of a disease. Examples of therapeutic agents include but are not limited to antibodies, antibody fragments, drugs, cytokine or chemokine inhibitors, proapoptotic agents, tyrosine kinase inhibitors, toxins, enzymes, nucleases, hormones, immunomodulators, antisense oligonucleotides, siRNA, RNAi, chelators, boron compounds, photoactive agents, dyes and radioisotopes.

A “diagnostic agent” is an atom, molecule, or compound that is useful in diagnosing a disease. Useful diagnostic agents include, but are not limited to, radioisotopes, dyes, contrast agents, fluorescent compounds or molecules and enhancing agents (e.g., paramagnetic ions). Preferably, the diagnostic agents are selected from the group consisting of radioisotopes, enhancing agents, and fluorescent compounds.

As used herein, a “radiolysis protection agent” refers to any molecule, compound or composition that may be added to an ¹⁸F- or ⁶⁸Ga-labeled complex or molecule to decrease the rate of breakdown of the labeled complex or molecule by radiolysis. Any known radiolysis protection agent, including but not limited to ascorbic acid, may be used.

¹⁸F Labeling Techniques

A variety of techniques for labeling molecules with ¹⁸F are known. Table 1 lists the properties of several of the more commonly reported fluorination procedures. Peptide labeling through carbon often involves ¹⁸F-binding to a prosthetic group through nucleophilic substitution, usually in 2- or 3-steps where the prosthetic group is labeled and purified, attached to the compound, and then purified again. This general method has been used to attach prosthetic groups through amide bonds, aldehydes, and “click” chemistry (Marik et al., 2006, Bioconj Chem 17:1017-21; Poethko et al., 2004, J Nucl Med 45:892-902; Li et al., 2007, Bioconj Chem 18:989-93). The most common amide bond-forming reagent has been N-succinimidyl 4-¹⁸F-fluorobenzoate (¹⁸F-SFB), but a number of other groups have been tested (Marik et al., 2006). In some cases, such as when ¹⁸F-labeled active ester amide-forming groups are used, it may be necessary to protect certain groups on a peptide during the coupling reaction, after which they are cleaved. The synthesis of this ¹⁸F-SFB reagent and subsequent conjugation to the peptide requires many synthetic steps and takes about 2-3 h.

A simpler, more efficient ¹⁸F-peptide labeling method was developed by Poethko et al. (2004), where a 4-¹⁸F-fluorobenzaldehyde reagent was conjugated to a peptide through an oxime linkage in about 75-90 min, including the dry-down step. The newer “click chemistry” method attaches ¹⁸F-labeled molecules onto peptides with an acetylene or azide in the presence of a copper catalyst (Li et al, 2007; Glaser and Arstad, 2007, Bioconj Chem 18:989-93). The reaction between the azide and acetylene groups forms a triazole connection, which is quite stable and forms very efficiently on peptides without the need for protecting groups. Click chemistry produces the ¹⁸F-labeled peptides in good yield (˜50%) in about 75-90 min with the dry-down step.

TABLE 1
Summary of selected ¹⁸F-labeling methods.

| | | Höhne et | | Glaser & | | |
|---|---|---|---|---|---|---|
| | Schirrmacher | al. | Li et al. | Arstad | Poethko et al. | Marik et |
| Author/Ref. | et al. (2007) | (2008) | (2007) | (2007) | (2004) | al (2006) |
| Attachment | Silicon | Silicon | Click | Click | Aldehyde/oxime | Amide |
| Rx steps | 2 | 1 | 2 | 2 | 2 | many |
| Rx time | 40 | 115-155 | 110 | 65-80 | 75-90 min | 110⁺ |
| (min)^{a} | | | | (estimated) | (estimated) | |
| Yield^{b} | 55% | 13% | 54% | 50% | 40% | 10% |
| HPLC- | 1 | 1 | 2 | 1 + | 1 | 2 |
| purification | | | | distillation | | |
| steps | | | | | | |
| Specific | 225-680 | 62 | high | high | high | high |
| Activity | | | | | | |
| (GBq/μmol) | | | | | | |

^{a}Including dry-down time
^{b}Decay corrected

A more recent method of binding ¹⁸F to silicon uses isotopic exchange to displace ¹⁹F with ¹⁸F (Shirrmacher et al., 2007). Performed at room temperature in 10 min, this reaction produces the ¹⁸F-prosthetic aldehyde group with high specific activity (225-680 GBq/μmol; 6,100-18,400 Ci/mmol). The ¹⁸F-labeled aldehyde is subsequently conjugated to a peptide and purified by HPLC, and the purified labeled peptide is obtained within 40 min (including dry-down) with ˜55% yield. This was modified subsequently to a single-step process by incorporating the silicon into the peptide before the labeling reaction (Hohne et al, 2008). However, biodistribution studies in mice with an ¹⁸F-silicon-bombesin derivative showed bone uptake increasing over time (1.35±0.47% injected dose (ID)/g at 0.5 h vs. 5.14±2.71% ID/g at 4.0 h), suggesting a release of ¹⁸F from the peptide, since unbound ¹⁸F is known to localize in bone (Hohne et al., 2008). HPLC analysis of urine showed a substantial amount of ¹⁸F activity in the void volume, which presumably is due to fluoride anion (¹⁸F) released from the peptide. It would therefore appear that the ¹⁸F-silicon labeled molecule was not stable in serum. Substantial hepatobiliary excretion was also reported, attributed to the lipophilic nature of the ¹⁸F-silicon-binding substrate, and requiring future derivatives to be more hydrophilic. Methods of attaching ¹⁸F to boron also have been explored; however, the current process produces conjugates with low specific activity (Ting et al., 2008).

Antibodies and peptides are coupled routinely with radiometals, typically in 15 min and in quantitative yields (Meares et al., 1984, Acc Chem Res 17:202-209; Scheinberg et al., 1982, Science 215:1511-13). For PET imaging, ⁶⁴Cu and ⁶⁸Ga have been bound to peptides via a chelate, and have shown reasonably good PET-imaging properties (Heppler et al., 2000, Current Med Chem 7:971-94). Since fluoride binds to most metals, we sought to determine if an ¹⁸F-metal complex could be bound to a chelator on a targeting molecule (Tewson, 1989, Nucl Med Biol. 16:533-51; Martin, 1996, Coordination Chem Rev 141:23-32). We have focused on the binding of an Al¹⁸F complex, since aluminum-fluoride can be relatively stable in vivo (Li, 2003, Crit Rev Oral Biol Med 14:100-114; Antonny et al., 1992, J Biol Chem 267:6710-18). Initial studies showed the feasibility of this approach to prepare an ¹⁸F-labeled peptide for in vivo targeting of cancer with a bispecific antibody (bsMAb) pretargeting system, a highly sensitive and specific technique for localizing cancer, in some cases better than [¹⁸F]FDG (fluorodeoxyglucose) (McBride et al., 2008, J Nucl Med (suppl) 49:97P; Wagner, 2008, J Nucl Med 49:23N-24N; Karacay et al., 2000, Bioconj Chem 11:842-54; Sharkey et al., 2008, Cancer Res 68; 5282-90; Gold Et al., 2008, Cancer Res 68:4819-26; Sharkey et al., 2005, Nature Med 11:1250-55; Sharkey et al., 2005, Clin Cancer Res 11:7109s-7121s; McBride et al., 2006, J Nucl Med 47:1678-88; Sharkey et al., 2008, Radiology 246:497-508). These studies revealed that an Al¹⁸F complex could bind stably to a 1,4,7-triazacyclononane-1,4,7-triacetic acid (NOTA), but the yields were low.

In the Examples below, new labeling conditions and several new chelating moieties were examined that enhanced yields from about 10% to about 80%, providing a feasible method for ¹⁸F-labeling of peptides and other molecules of use in PET imaging.

Detection of Prostate Cancer Using Labeled GRPR Antagonists

Prostate cancer (PC) is the most frequently diagnosed cancer and the second leading cause of cancer death among men in the United States (Siegel et al., 2012, CA 62:10-29). There is a strong need for improved imaging techniques that provide accurate staging and monitoring of this disease. Conventional diagnostic techniques, such as ultrasound-guided biopsy, are limited by high false-negative rates (Roehl et al., 2002, J Urol 167:2435-2439). Emerging functional imaging techniques, including diffusion-weighted magnetic resonance (DW-MR) imaging, dynamic contrast-enhanced MR (DCE-MR) imaging and positron emission tomography (PET), have shown improved sensitivity and staging accuracy for detecting primary prostate tumors and metastatic lymph nodes (Talab et al., 2012, Radiol Clin North Am 50:1015-1041).

Several PET radiotracers have shown promising clinical utility, such as the metabolic agents ¹⁸F-FDG, ¹¹C/¹⁸F-choline and ¹¹C/¹⁸F-acetate, for the assessment of distant metastasis, and ¹⁸F-NaF for the detection of bone metastasis (Mari Aparici & Seo, 2012, Semin Nucl Med 42:328-342). However, their application seems to be limited to late stage, recurrent or metastatic prostate cancer. Increasing effort is being made in developing PET imaging agents targeting specific biomarkers of prostate cancer, such as gastrin-releasing peptide receptor (GRPR) (for review, see Sancho et al., 2011, Curr Drug Deliv 8:79-134) and prostate-specific membrane antigen (PSMA) (for review, see Osborne et al., 2013, Urol Oncol 31:144-154.).

The gastrin-releasing peptide receptor, also named bombesin receptor subtype II, has been shown to be over-expressed in several human tumors, including prostate cancer (Reubi et al., 2002, Clin Cancer Res 8:1139-1146). Over-expression of GRPR was found in 63-100% of prostate primary tumors and over 50% of lymph and bone metastases (Ananias et al., 2009, Prostate 69:1101-1108). Because of their low expression in benign prostatic hyperplasia and inflammatory prostatic tissues, imaging of GRPR has potential advantages over choline- and acetate-based radiotracers (Markwalder et al., 1999, Cancer Res 59:1152-1159; Beer et al., 2012, Prostate 72:318-325).

A variety of radiolabeled bombesin analogs have been developed for targeting GRPR-positive tumors and were evaluated in preclinical and clinical studies (Sancho et al., 2011, Curr Drug Deliv 8:79-134). Several studies have shown that GRPR antagonists show superior properties over GRPR agonists, affording higher tumor uptake and lower accumulation in physiological GRPR-positive non-target tissues (Cescato et al., 2008, J Nucl Med 49:318-326; Mansi et al., 2009, Clin Cancer Res 15:5240-5249). In addition, GRPR agonists were shown to stimulate tumor growth and angiogenesis (Cuttitta et al., 1985, Nature 316:823-826; Schally et al., 2001, Front Neuroendocrinol 22:248-291) and induced side effects in patients mediated by their physiological activity (Basso et al. World J Surg 3:579-585; Bodei et al., 2007, Eur J Nucl Med Mol Imaging 34:S221-S221). Therefore, particular attention has been drawn to the development of GRPR antagonists for imaging and radionuclide therapy of prostate cancer. Several GRPR antagonists have been developed in the past by the modification of C-terminal residues of GRPR agonists, including the statin-based bombesin analog JMV594 (Llinares et al., 1999, J Pept Res 53:275-283).

⁶⁸Ga-labeled GRPR antagonists were developed for PET imaging, showing good targeting properties in preclinical studies (Mansi et al., 2009, Clin Cancer Res 15:5240-5249; Mansi et al., 2011, Eur J Nucl Med Mol imaging 38:97-107; Abiraj et al., 2011, J Nucl Med 52:1970-1978; Varasteh et al., 2013, Bioconj Chem 24:1144-1153), and recently also in clinical studies (Roivainen et al., 2013, J Nucl Med 54:867-872; Kahkonen et al., 2013, Clin Cancer Res 19:5434-5443). Clinical evaluation of the ⁶⁸Ga-labeled GRPR antagonist (BAY86-7548) has shown superior accuracy (83%) of this tracer in comparison to the currently used ¹⁸F/¹¹C-labeled acetate and choline in detection of primary prostate cancer. However the detection of lymph node metastases with this tracer was suboptimal, partially due to the suboptimal physical characteristics of ⁶⁸Ga compared to ¹⁸F, limiting the detection of small lesions (Kahkonen et al., 2013, Clin Cancer Res 19:5434-5443). One aim of the present study was to develop an ¹⁸F-labeled GRPR antagonist for high resolution and sensitive PET imaging of primary, recurrent and metastatic prostate cancer, and compare the imaging properties of this tracer with those of ⁶⁸Ga-labeled analogs.

¹⁸F has superior physical characteristics for PET imaging, such as a lower positron range and a higher positron yield, offering higher resolution and sensitivity (Sanchez-Crespo, 2013, Appl Radiat Isot 76:55-62). Most methods for labeling peptides with ¹⁸F are laborious and require multi-step procedures with moderate labeling yields. A good alternative is the Al¹⁸F labeling method (McBride et al., 2009, J Nucl Med 50:991-998), allowing fast and facile labeling of peptides in a one-step procedure. We have designed a new GRPR-antagonist conjugate, analogous to the previously described JMV4168 [DOTA-βAla₂-JMV594] (Marsouvanidis et al., 2013, J Med Chem 56:2374-2384), with a NODA-MPAA chelator (JMV5132) for high-yield complexation of Al¹⁸F. In Example 1 below, we report on the direct preclinical comparison of this novel radiolabeled tracer with ⁶⁸Ga-JMV4168 and ⁶⁸Ga-JMV5132 as reference, for PET imaging of prostate cancer. We determined the in vitro characteristics of the radiolabeled peptides, and evaluated their tumor targeting properties in nude mice with subcutaneous tumors. The results demonstrate the utility of ¹⁸F-labeled GRPR antagonists for early detection and/or diagnosis of prostate cancer and other GRPR-expressing tumors.

Chelating Moieties

In some embodiments, an ¹⁸F-, ¹⁹F- or ⁶⁸Ga-labeled molecule may comprise one or more hydrophilic chelating moieties, which can bind metal ions and also help to ensure rapid in vivo clearance. Chelators may be selected for their particular metal-binding properties, and may be readily interchanged.

Particularly useful metal-chelate combinations include 2-benzyl-DTPA and its monomethyl and cyclohexyl analogs. Macrocyclic chelators such as NOTA (1,4,7-triazacyclononane-1,4,7-triacetic acid), DOTA, TETA (p-bromoacetamido-benzyl-tetraethylaminetetraacetic acid) and NETA are also of use with a variety of metals, that may potentially be used as ligands for ¹⁸F- or ¹⁹F-labeling.

DTPA and DOTA-type chelators, where the ligand includes hard base chelating functions such as carboxylate or amine groups, are most effective for chelating hard acid cations, especially Group IIa and Group IIIa metal cations. Such metal-chelate complexes can be made very stable by tailoring the ring size to the metal of interest. Other ring-type chelators such as macrocyclic polyethers are of interest for stably binding nuclides. Porphyrin chelators may be used with numerous metal complexes. More than one type of chelator may be conjugated to a carrier to bind multiple metal ions. Chelators such as those disclosed in U.S. Pat. No. 5,753,206, especially thiosemicarbazonylglyoxylcysteine (Tscg-Cys) and thiosemicarbazinyl-acetylcysteine (Tsca-Cys) chelators are advantageously used to bind soft acid cations of Tc, Re, Bi and other transition metals, lanthanides and actinides that are tightly bound to soft base ligands. It can be useful to link more than one type of chelator to a peptide. Because antibodies to a di-DTPA hapten are known (Barbet et al., U.S. Pat. Nos. 5,256,395) and are readily coupled to a targeting antibody to form a bispecific antibody, it is possible to use a peptide hapten with cold diDTPA chelator and another chelator for binding an ¹⁸F complex, in a pretargeting protocol. One example of such a peptide is Ac-Lys(DTPA)-Tyr-Lys(DTPA)-Lys(Tscg-Cys)-NH₂ (core peptide disclosed as SEQ ID NO:1). Other hard acid chelators such as DOTA, TETA and the like can be substituted for the DTPA and/or Tscg-Cys groups, and MAbs specific to them can be produced using analogous techniques to those used to generate the anti-di-DTPA MAb.

Another useful chelator may comprise a NOTA-type moiety, for example as disclosed in Chong et al. (J. Med. Chem., 2008, 51:118-25). Chong et al. disclose the production and use of a bifunctional C-NETA ligand, based upon the NOTA structure, that when complexed with ¹⁷⁷Lu or ^{205/206}Bi showed stability in serum for up to 14 days. The chelators are not limiting and these and other examples of chelators that are known in the art and/or described in the following Examples may be used in the practice of the invention.

It will be appreciated that two different hard acid or soft acid chelators can be incorporated into the targeting peptide, e.g., with different chelate ring sizes, to bind preferentially to two different hard acid or soft acid cations, due to the differing sizes of the cations, the geometries of the chelate rings and the preferred complex ion structures of the cations. This will permit two different metals, one of which may be attached to ¹⁸F and the other to ⁶⁸Ga, to be incorporated into a targeting peptide.

Peptides

The targeting peptides used are conveniently synthesized on an automated peptide synthesizer using a solid-phase support and standard techniques of repetitive orthogonal deprotection and coupling. Free amino groups in the peptide, that are to be used later for conjugation of chelating moieties or other agents, are advantageously blocked with standard protecting groups such as a Boc group, while N-terminal residues may be acetylated to increase serum stability. Such protecting groups are well known to the skilled artisan. See Greene and Wuts Protective Groups in Organic Synthesis, 1999 (John Wiley and Sons, N.Y.). Peptides are advantageously cleaved from the resins to generate the corresponding C-terminal amides, in order to inhibit in vivo carboxypeptidase activity. Exemplary structures of use and methods of peptide synthesis are disclosed in the Examples below. Chelating moieties may be conjugated to peptides using bifunctional chelating moieties as discussed below.

Amino Acid Substitutions

Certain embodiments may involve production and use of targeting peptides with one or more substituted amino acid residues. The skilled artisan will be aware that amino acid substitutions typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within ±2 is preferred, within ±1 are more preferred, and within ±0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5 .+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Some embodiments may involve substitution of one or more D-amino acids for the corresponding L-amino acids. Peptides comprising D-amino acid residues are more resistant to peptidase activity than L-amino acid comprising peptides. Such substitutions may be readily performed using standard amino acid synthesizers, as discussed in the Examples below.

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded protein sequence are well known and a matter of routine experimentation for the skilled artisan, for example by the technique of site-directed mutagenesis or by synthesis and assembly of oligonucleotides encoding an amino acid substitution and splicing into an expression vector construct.

Click Chemistry

In various embodiments, targeting peptide conjugates may be prepared using click chemistry technology. The click chemistry approach was originally conceived as a method to rapidly generate complex substances by joining small subunits together in a modular fashion. (See, e.g., Kolb et al., 2004, Angew Chem Int Ed 40:3004-31; Evans, 2007, Aust J Chem 60:384-95.) Various forms of click chemistry reaction are known in the art, such as the Huisgen 1,3-dipolar cycloaddition copper catalyzed reaction (Tornoe et al., 2002, J Organic Chem 67:3057-64), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

The azide alkyne Huisgen cycloaddition reaction uses a copper catalyst in the presence of a reducing agent to catalyze the reaction of a terminal alkyne group attached to a first molecule. In the presence of a second molecule comprising an azide moiety, the azide reacts with the activated alkyne to form a 1,4-disubstituted 1,2,3-triazole. The copper catalyzed reaction occurs at room temperature and is sufficiently specific that purification of the reaction product is often not required. (Rostovstev et al., 2002, Angew Chem Int Ed 41:2596; Tornoe et al., 2002, J Org Chem 67:3057.) The azide and alkyne functional groups are largely inert towards biomolecules in aqueous medium, allowing the reaction to occur in complex solutions. The triazole formed is chemically stable and is not subject to enzymatic cleavage, making the click chemistry product highly stable in biological systems. However, the copper catalyst is toxic to living cells, precluding biological applications.

A copper-free click reaction has been proposed for covalent modification of biomolecules in living systems. (See, e.g., Agard et al., 2004, J Am Chem Soc 126:15046-47.) The copper-free reaction uses ring strain in place of the copper catalyst to promote a [3+2] azide-alkyne cycloaddition reaction (Id.) For example, cyclooctyne is a 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions, without the toxic copper catalyst (Id.)

Another type of copper-free click reaction was reported by Ning et al. (2010, Angew Chem Int Ed 49:3065-68), involving strain-promoted alkyne-nitrone cycloaddition. To address the slow rate of the original cyclooctyne reaction, electron-withdrawing groups are attached adjacent to the triple bond (Id.) Examples of such substituted cyclooctynes include difluorinated cyclooctynes, 4-dibenzocyclooctynol and azacyclooctyne (Id.) An alternative copper-free reaction involved strain-promoted alkyne-nitrone cycloaddition to give N-alkylated isoxazolines (Id.) The reaction was reported to have exceptionally fast reaction kinetics and was used in a one-pot three-step protocol for site-specific modification of peptides and proteins (Id.) Nitrones were prepared by the condensation of appropriate aldehydes with N-methylhydroxylamine and the cycloaddition reaction took place in a mixture of acetonitrile and water (Id.)

The Diels-Alder reaction has also been used for in vivo labeling of molecules. Rossin et al. (2010, Angew Chem Int Ed 49:3375-78) reported a 52% yield in vivo between a tumor-localized anti-TAG72 (CC49) antibody carrying a trans-cyclooctene (TCO) reactive moiety and an ^{lll}In-labeled tetrazine DOTA derivative. The TCO-labeled CC49 antibody was administered to mice bearing colon cancer xenografts, followed 1 day later by injection of ^{lll}In-labeled tetrazine probe (Id.) The reaction of radiolabeled probe with tumor localized antibody resulted in pronounced radioactivity localized in the tumor, as demonstrated by SPECT imaging of live mice three hours after injection of radiolabeled probe, with a tumor-to-muscle ratio of 13:1 (Id.) The results confirmed the in vivo chemical reaction of the TCO and tetrazine-labeled molecules.

Modifications of click chemistry reactions are suitable for use in vitro or in vivo. Reactive targeting molecule may be formed either by either chemical conjugation or by biological incorporation. The targeting peptide may be activated with an azido moiety, a substituted cyclooctyne or alkyne group, or a nitrone moiety. Where the targeting peptide comprises an azido or nitrone group, the corresponding chelator will comprise a substituted cyclooctyne or alkyne group, and vice versa. Such activated molecules may be made by metabolic incorporation in living cells, as discussed above. Alternatively, methods of chemical conjugation of such moieties to biomolecules are well known in the art, and any such known method may be utilized. The disclosed techniques may be used in combination with the diagnostic radionuclide (e.g., ¹⁸F) labeling methods described below for PET, SPECT or MRI imaging, or alternatively may be utilized for delivery of any therapeutic and/or diagnostic agent that may be attached to a suitable activated targeting peptide.

Therapeutic Agents

In various embodiments, the labeled targeting peptides may be administered in combination with one or more additional therapeutic or diagnostic agents. Such additional agents may be administered before, concurrently with, or after the labeled peptide. Therapeutic agents of use may include cytotoxic agents, anti-angiogenic agents, pro-apoptotic agents, antibiotics, hormones, hormone antagonists, chemokines, drugs, prodrugs, toxins, enzymes, antibodies, antibody fragments, immunoconjugates, immunomodulators, oligonucleotides, siRNA, RNAi or other known agents.

Drugs of use may possess a pharmaceutical property selected from the group consisting of antimitotic, antikinase, alkylating, antimetabolite, antibiotic, alkaloid, anti-angiogenic, pro-apoptotic agents and combinations thereof. Exemplary drugs of use include, but are not limited to, 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, Cox-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, doxorubicin, 2-pyrrolinodoxorubicine (2P-DOX), pro-2P-DOX, cyano-morpholino doxorubicin, doxorubicin glucuronide, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, navelbine, neratinib, nilotinib, nitrosurea, olaparib, plicomycin, procarbazine, paclitaxel, PCI-32765, pentostatin, PSI-341, raloxifene, semustine, sorafenib, streptozocin, SU11248, sunitinib, tamoxifen, temazolomide (an aqueous form of DTIC), transplatinum, thalidomide, thioguanine, thiotepa, teniposide, topotecan, uracil mustard, vatalanib, vinorelbine, vinblastine, vincristine, vinca alkaloids and ZD1839.

Toxins of use may include ricin, abrin, alpha toxin, saporin, ribonuclease (RNase), e.g., onconase, DNase I, Staphylococcal enterotoxin-A, pokeweed antiviral protein, gelonin, diphtheria toxin, Pseudomonas exotoxin, and Pseudomonas endotoxin.

Chemokines of use may include RANTES, MCAF, MIP1-alpha, MIP1-Beta and IP-10.

In certain embodiments, anti-angiogenic agents, such as angiostatin, baculostatin, canstatin, maspin, anti-VEGF antibodies, anti-PlGF peptides and antibodies, anti-vascular growth factor antibodies, anti-Flk-1 antibodies, anti-Flt-1 antibodies and peptides, anti-Kras antibodies, anti-cMET antibodies, anti-MIF (macrophage migration-inhibitory factor) antibodies, laminin peptides, fibronectin peptides, plasminogen activator inhibitors, tissue metalloproteinase inhibitors, interferons, interleukin-12, IP-10, Gro-β, thrombospondin, 2-methoxyoestradiol, proliferin-related protein, carboxiamidotriazole, CM101, Marimastat, pentosan polysulphate, angiopoietin-2, interferon-alpha, herbimycin A, PNU145156E, 16K prolactin fragment, Linomide (roquinimex), thalidomide, pentoxifylline, genistein, TNP-470, endostatin, paclitaxel, accutin, angiostatin, cidofovir, vincristine, bleomycin, AGM-1470, platelet factor 4 or minocycline may be of use.

Immunomodulators of use may be selected from a cytokine, a stem cell growth factor, a lymphotoxin, a hematopoietic factor, a colony stimulating factor (CSF), an interferon (IFN), erythropoietin, thrombopoietin and a combination thereof. Specifically useful are lymphotoxins such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF), interferon, such as interferons-α, -β or -γ, and stem cell growth factor, such as that designated “S1 factor”. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-25, LIF, kit-ligand or FLT-3, angiostatin, thrombospondin, endostatin, tumor necrosis factor and LT.

Radionuclides of use include, but are not limited to-¹¹¹In, ¹⁷⁷Lu, ²¹²Bi, ²¹³Bi, ²¹¹At, ⁶²Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, ²²⁷Th, and ²¹¹Pb. The therapeutic radionuclide preferably has a decay-energy in the range of 20 to 6,000 keV, preferably in the ranges 60 to 200 keV for an Auger emitter, 100-2,500 keV for a beta emitter, and 4,000-6,000 keV for an alpha emitter. Maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, 1-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213, Th-227 and Fm-255. Decay energies of useful alpha-particle-emitting radionuclides are preferably 2,000-10,000 keV, more preferably 3,000-8,000 keV, and most preferably 4,000-7,000 keV. Additional potential radioisotopes of use include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ¹⁰³Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like. Some useful diagnostic nuclides may include ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ⁹⁴Tc, ^{94m}Tc, ^{99m}Tc, or ¹¹¹In.

Therapeutic agents may include a photoactive agent or dye. Fluorescent compositions, such as fluorochrome, and other chromogens, or dyes, such as porphyrins sensitive to visible light, have been used to detect and to treat lesions by directing the suitable light to the lesion. In therapy, this has been termed photoradiation, phototherapy, or photodynamic therapy. See Joni et al. (eds.), PHOTODYNAMIC THERAPY OF TUMORS AND OTHER DISEASES (Libreria Progetto 1985); van den Bergh, Chem. Britain (1986), 22:430. Moreover, targeting molecules have been coupled with photoactivated dyes for achieving phototherapy. See Mew et al., J. Immunol. (1983),130:1473; idem., Cancer Res. (1985), 45:4380; Oseroff et al., Proc. Natl. Acad. Sci. USA (1986), 83:8744; idem., Photochem. Photobiol. (1987), 46:83; Hasan et al., Prog. Clin. Biol. Res. (1989), 288:471; Tatsuta et al., Lasers Surg. Med. (1989), 9:422; Pelegrin et al., Cancer (1991), 67:2529.

Other useful therapeutic agents may comprise oligonucleotides, especially antisense oligonucleotides that preferably are directed against oncogenes and oncogene products, such as bcl-2 or p53. A preferred form of therapeutic oligonucleotide is siRNA.

Diagnostic Agents

Diagnostic agents are preferably selected from the group consisting of a radionuclide, a radiological contrast agent, a paramagnetic ion, a metal, a fluorescent label, a chemiluminescent label, an ultrasound contrast agent and a photoactive agent. Such diagnostic agents are well known and any such known diagnostic agent may be used. Non-limiting examples of diagnostic agents may include a radionuclide such as ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ^{52m}Mn, ⁵⁵Co, ⁷²As, ⁷⁵Br, ⁷⁶Br, ^{82m}Rb, ⁸³Sr, or other gamma-, beta-, or positron-emitters. Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III). Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoerytherin, phycocyanin, allophycocyanin, o-phthaldehyde and fluorescamine. Chemiluminescent labels of use may include luminol, isoluminol, an aromatic acridinium ester, an imidazole, an acridinium salt or an oxalate ester.

Methods of Administration

The labeled targeting peptides may be formulated to obtain compositions that include one or more pharmaceutically suitable excipients, one or more additional ingredients, or some combination of these. These can be accomplished by known methods to prepare pharmaceutically useful dosages, whereby the active ingredients (i.e., the labeled peptides) are combined in a mixture with one or more pharmaceutically suitable excipients. Sterile phosphate-buffered saline is one example of a pharmaceutically suitable excipient. Other suitable excipients are well known to those in the art. See, e.g., Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The preferred route for administration of the compositions described herein is parenteral injection. Injection may be intravenous, intraarterial, intralymphatic, intrathecal, subcutaneous or intracavitary (i.e., parenterally). In parenteral administration, the compositions will be formulated in a unit dosage injectable form such as a solution, suspension or emulsion, in association with a pharmaceutically acceptable excipient. Such excipients are inherently nontoxic and nontherapeutic. Examples of such excipients are saline, Ringer's solution, dextrose solution and Hank's solution. Nonaqueous excipients such as fixed oils and ethyl oleate may also be used. A preferred excipient is 5% dextrose in saline. The excipient may contain minor amounts of additives such as substances that enhance isotonicity and chemical stability, including buffers and preservatives. Other methods of administration, including oral administration, are also contemplated.

Formulated compositions comprising labeled targeting peptides can be used for intravenous administration via, for example, bolus injection or continuous infusion. Compositions for injection can be presented in unit dosage form, e.g., in ampoules or in multi-dose containers, with an added preservative. Compositions can also take such forms as suspensions, solutions or emulsions in oily or aqueous vehicles, and can contain formulatory agents such as suspending, stabilizing and/or dispersing agents. Alternatively, the compositions can be in powder form for constitution with a suitable vehicle, e.g., sterile pyrogen-free water, before use.

The compositions may be administered in solution. The pH of the solution should be in the range of pH 5 to 9.5, preferably pH 6.5 to 7.5. The formulation thereof should be in a solution having a suitable pharmaceutically acceptable buffer such as phosphate, TRIS (hydroxymethyl) aminomethane-HCl or citrate and the like. In certain preferred embodiments, the buffer is potassium biphthalate (KHP), which may act as a transfer ligand to facilitate ¹⁸F-, ¹⁹F- or ⁶⁸Ga-labeling. Buffer concentrations should be in the range of 1 to 100 mM. The formulated solution may also contain a salt, such as sodium chloride or potassium chloride in a concentration of 50 to 150 mM. An effective amount of a stabilizing agent such as glycerol, albumin, a globulin, a detergent, a gelatin, a protamine or a salt of protamine may also be included. The compositions may be administered to a mammal subcutaneously, intravenously, intramuscularly or by other parenteral routes. Moreover, the administration may be by continuous infusion or by single or multiple boluses.

In general, the dosage of ¹⁸F, ⁶⁸Ga or other radiolabel to administer to a human subject will vary depending upon such factors as the patient's age, weight, height, sex, general medical condition and previous medical history. Preferably, a saturating dose of the labeled molecule is administered to a patient. For administration of radiolabeled molecules, the dosage may be measured by millicuries. A typical range for imaging studies would be five to 10 mCi.

Administration of Peptides

Various embodiments of the claimed methods and/or compositions may concern one or more ¹⁸F-, ¹⁹F- or ⁶⁸Ga-labeled peptides to be administered to a subject. Administration may occur by any route known in the art, including but not limited to oral, nasal, buccal, inhalational, rectal, vaginal, topical, orthotopic, intradermal, subcutaneous, intramuscular, intraperitoneal, intraarterial, intrathecal or intravenous injection.

In certain embodiments, the standard peptide bond linkage may be replaced by one or more alternative linking groups, such as CH₂—NH, CH₂—S, CH₂—CH₂, CH═CH, CO—CH₂, CHOH—CH₂ and the like. Methods for preparing peptide mimetics are well known (for example, Hruby, 1982, Life Sci 31:189-99; Holladay et al., 1983, Tetrahedron Lett. 24:4401-04; Jennings-White et al., 1982, Tetrahedron Lett. 23:2533; Almquiest et al., 1980, J. Med. Chem. 23:1392-98; Hudson et al., 1979, Int. J. Pept. Res. 14:177-185; Spatola et al., 1986, Life Sci 38:1243-49; U.S. Pat. Nos. 5,169,862; 5,539,085; 5,576,423, 5,051,448, 5,559,103.) Peptide mimetics may exhibit enhanced stability and/or absorption in vivo compared to their peptide analogs.

Peptide stabilization may also occur by substitution of D-amino acids for naturally occurring L-amino acids, particularly at locations where endopeptidases are known to act. Endopeptidase binding and cleavage sequences are known in the art and methods for making and using peptides incorporating D-amino acids have been described (e.g., U.S. Patent Application Publication No. 20050025709, McBride et al., filed Jun. 14, 2004, the Examples section of which is incorporated herein by reference).

Imaging Using Labeled Molecules

Methods of imaging using labeled molecules are well known in the art, and any such known methods may be used with the labeled targeting peptides disclosed herein. See, e.g., U.S. Pat. Nos. 6,241,964; 6,358,489; 6,953,567 and published U.S. Patent Application Publ. Nos. 20050003403; 20040018557; 20060140936, the Examples section of each incorporated herein by reference. See also, Page et al., Nuclear Medicine And Biology, 21:911-919, 1994; Choi et al., Cancer Research 55:5323-5329, 1995; Zalutsky et al., J. Nuclear Med., 33:575-582, 1992; Woessner et. al. Magn. Reson. Med. 2005, 53: 790-99.

Methods of diagnostic imaging with labeled peptides are well-known. For example, in the technique of immunoscintigraphy, peptide ligands are labeled with a gamma-emitting radioisotope and introduced into a patient. A gamma camera is used to detect the location and distribution of gamma-emitting radioisotopes. See, for example, Srivastava (ed.), RADIOLABELED MONOCLONAL ANTIBODIES FOR IMAGING AND THERAPY (Plenum Press 1988), Chase, “Medical Applications of Radioisotopes,” in REMINGTON'S PHARMACEUTICAL SCIENCES, 18th Edition, Gennaro et al. (eds.), pp. 624-652 (Mack Publishing Co., 1990), and Brown, “Clinical Use of Monoclonal Antibodies,” in BIOTECHNOLOGY AND PHARMACY 227-49, Pezzuto et al. (eds.) (Chapman & Hall 1993). Also preferred is the use of positron-emitting radionuclides (PET isotopes), such as with an energy of 511 keV, such as ¹⁸F, ⁶⁸Ga, ⁶⁴Cu, and ¹²⁴I. Such radionuclides may be imaged by well-known PET scanning techniques.

Kits

Various embodiments may concern kits containing components suitable for imaging, diagnosing and/or detecting diseased tissue in a patient using labeled compounds. Exemplary kits may contain a targeting peptide of use as described herein.

A device capable of delivering the kit components may be included. One type of device, for applications such as parenteral delivery, is a syringe that is used to inject the composition into the body of a subject. Inhalation devices may also be used for certain applications.

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers. Another component that can be included is instructions to a person using a kit for its use.

### EXAMPLES

### Example 1

### Comparison of Al¹⁸F- and ⁶⁸Ga-Labeled GRPR-Antagonists for PET Imaging of Prostate Cancer

Summary

Bombesin (BBN) analog Gastrin-releasing peptide receptor (GRPR) which is overexpressed in human prostate cancer (PC), has been successfully used as target for molecular imaging of PC. In this study, we report on the direct comparison of 3 novel GRPR-targeted radiolabeled tracers: Al¹⁸F-JMV5132, ⁶⁸Ga-JMV4168 and ⁶⁸Ga-JMV5132. Methods: The GRPR antagonist JMV594 [H-d-Phe-Gln-Trp-Ala-Val-Gly-His-Sta-Leu-NH₂] (SEQ ID NO: 2) was conjugated to NODA-MPAA for labeling with Al¹⁸F. JMV5132 [NODA-MPAA-(βAla)₂-JMV594] (SEQ ID NO: 5) was radiolabeled with ⁶⁸Ga and Al¹⁸F, JMV4168 [DOTA-(βAla)₂-JMV594] (SEQ ID NO: 6) was labeled with ⁶⁸Ga for comparison. The IC₅₀ values for binding GRPR of JMV4168, JMV5132, ^{nat}Ga-JMV4168 and ^{nat}Ga-JMV5132 were determined in a competition-binding assay using GRPR overexpressing PC-3 tumors. The tumor targeting characteristics of the compounds were assessed in mice bearing subcutaneous (s.c.) PC-3 xenografts. Small-animal PET/CT images were acquired and tracer biodistribution was determined by ex-vivo measurements. Results: JMV5132 was labeled with ¹⁸F in a novel one-pot one-step procedure, within 20 min, without need for further purification and with a specific activity of 35 MBq/nmol. The log P values of ⁶⁸Ga-JMV4168, ⁶⁸Ga-JMV5132 and Al¹⁸F-JMV5132 were −2.53±0.04, −1.40±0.01 and −1.56±0.08 respectively. IC₅₀ values [in nM (95% confidence interval)] of JMV5132, JMV4168, ^{nat}Ga-JMV5132 and ^{nat}Ga-JMV4168 were 6.8 (4.6-10.0), 13.2 (5.9-29.3), 3.0 (1.5-6.0), and 3.2 (1.8-5.9), respectively. In mice with s.c. PC-3 xenografts all tracers cleared rapidly from the blood; exclusively via the kidney for ⁶⁸Ga-JMV4168, and partially hepatobiliary for ⁶⁸Ga-JMV5132 and Al¹⁸F-JMV5132. Two hours after injection, the uptake of⁶⁸Ga-JMV4168, ⁶⁸Ga-JMV5132 and Al¹⁸F-JMV5132 in PC-3 tumors was 5.96±1.39, 5.24±0.29, 5.30±0.98 (in % ID/g), respectively. GRPR-specificity was demonstrated by significantly reduced tumor uptake of⁶⁸Ga-JMV4168, ⁶⁸Ga-JMV5132 and Al¹⁸F-JMV5132 after co-injection of 100-fold excess of unlabeled JMV4168 or JMV5132. PET/CT clearly visualized PC-3 tumors with the highest resolution for Al¹⁸F-JMV5132. Conclusion: JMV5132 could be rapidly and efficiently labeled with ¹⁸F without need for further purification. ⁶⁸Ga-JMV4168, ⁶⁸Ga-JMV5132 and Al¹⁸F-JMV5132 showed high and specific accumulation in the GRPR-positive PC-3 tumors. Higher resolution PET images could be obtained with Al¹⁸F-JMV5132 with only low hepatobiliary excretion.

Materials and Methods

Synthesis of MV4168 and JMV5132

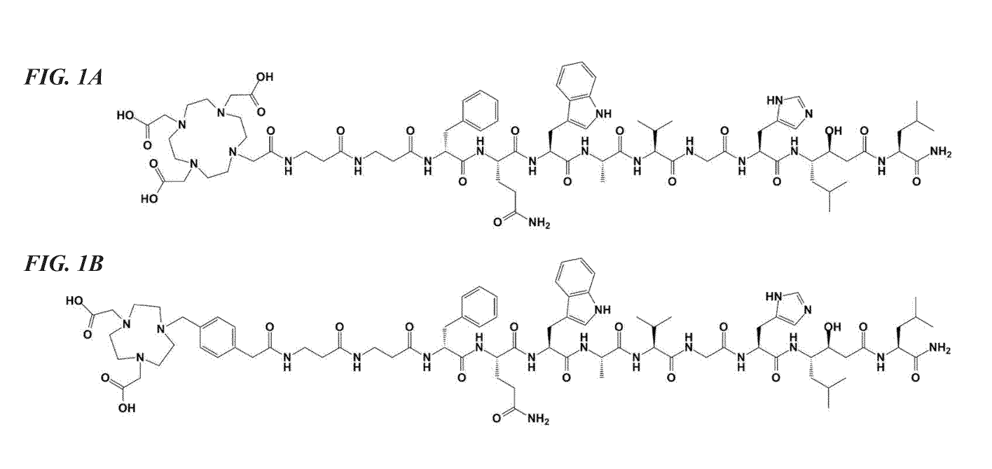

JMV4168 [DOTA-(βAla)₂-JMV594] (SEQ ID NO: 6) was synthesized using Fmoc-based solid-phase peptide synthesis as described previously (Marsouvanidis et al., 2013, J Med Chem 56:2374-2384). JMV5132 [NODA-MPAA-(βAla)₂-JMV594] (SEQ ID NO: 5) was synthesized like JMV4168 but was coupled to tert-butyl (tBu) protected NODA-MPAA instead of tBu-protected DOTA. NODA-MPAA was prepared as previously described using NO2AtBu (Chematech, Dijon, France) (D'Souza et al., 2011, Bioconj Chem 22:1793-1803). Chemical structures of JMV4168 and JMV5132 are shown in FIG. 1A and FIG. 1B.

Radiolabeling

Concentration and purification of ¹⁸F^{- 18}F⁻ solution in water (BV Cyclotron, VU, Amsterdam) was purified from metal impurities and concentrated before use. A CM cartridge (Waters, Sep-Pak Accell Plus CM, 130 mg) and a QMA cartridge (Waters, Sep-Pak Waters Accell Plus QMA Plus Light, 130 mg) were pre-washed with 10 mL metal-free deionized (DI) water. The ¹⁸F⁻ solution (8-15 GBq) was pushed slowly through the CM cartridge connected to the QMA cartridge, followed by 6 mL wash with metal-free DI water. Finally, ¹⁸F⁻ was eluted from the QMA cartridge in a small volume (150-200 μL)of saline.

Radiolabeling of JMV5132 with Al¹⁸F⁻ Labeling was performed by mixing ¹⁸F⁻ purified solution (15-20 μL, 700-900 MBq), NaOAc (0.5 μL of 1 M solution, pH 4.1), Al³⁺ stock solution (20 nmol, 10 μl of 2 mM AlCl₃.6H₂O in 0.1 M NaOAc, pH 4.2), acetonitrile (67% v/v), quenchers (2.5 μL of 50 mM methionine, gentisic acid and ascorbic acid), and finally JMV5132 (20 nmol, 3.26 μL of 10 μg/μL solution in 2 mM NaOAc pH 4.1). The reaction mixture was heated for 15 min at 105° C. For injection into mice, the peptide was diluted to less than 0.5% (v/v) acetonitrile with 0.5% (w/v) bovine serum albumin (BSA), 0.5% (w/v) Tween-20, and quenchers (1 mM methionine, gentisic acid and ascorbic acid) in phosphate-buffered saline (PBS), pH 7.4.

Radiolabeling of JMV4168 and JMV5132 with ⁶⁸Ga Elution and purification of ⁶⁸Ga from a ⁶⁸Ga/⁶⁸Ge generator (IGC-100, Eckert & Ziegler Europe) was performed using the NaCl-based procedure described earlier (Mueller et al., 2012, Bioconj Chem 23:1712-1717). 375 μL of HEPES (1 M, pH 3.6) was slowly added to 300 μL of purified ⁶⁸Ga eluate, followed by addition of quenchers (methionine, gentisic acid and ascorbic acid, 1.25 mM) and peptide (2 nmol). The reaction mixture was heated for 10 min at 95° C. After reaction, EDTA (50 mM) was added to a final concentration of 5 mM to complex free ⁶⁸Ga. For animal experiments, the labeled product was purified by RP-HPLC using the gradient described in the “Quality Control” section and concentrated by evaporation. For injection into mice, the radiolabeled peptide was diluted with 0.5% (w/v) BSA, 0.5% (w/v) Tween-20, and quenchers (1 mM methionine, gentisic acid and ascorbic acid) in PBS, and neutralized with NaHCO₃ buffer (1 M, pH 8.5).

Cold labeling of JMV4168 and JMV5132 with ^{nat}Ga. 2 μL Ga(NO₃)₃ solution (0.2 M) was added to 10 μL of HEPES (1 M, pH 3.6), followed by addition of quenchers (methionine, gentisic acid and ascorbic acid, 5 mM) and peptide (100 nmol). Reaction mixture was heated for 10 min at 95° C.

Quality Control

Peptide synthesis Purification of JMV5132 was accomplished by a preparative HPLC (Waters Delta Preparative, Waters 4000 system controller) with a C18 column (40 mm×100 mm, Waters DELTA PAK™, column II). Final product was characterized by RP-HPLC (Beckman, LC-126) on a reverse phase-18 CHROMOLITH® SpeedROD column (50 mm×4.6 mm, Merck, column I) and ESI/MS (Waters micromass ZQ, Waters 2695 Separation Module).

Stability of the radiolabeled peptides was analyzed by RP-HPLC after incubation in reaction mixture (2 h at room temperature) or human serum (2 h at 37° C.), in the presence or absence of added quenchers (1 mM methionine, gentisic acid and ascorbic acid).

Radiolabeling Labeling efficiency and colloid formation was assessed by iTLC using silica paper (Agilent) and 0.1 M NH₄OAc pH 5.5: 0.1 M EDTA (1:1), or 1 M NH₄OAc:MeOH (1:3), respectively. Radiochemical purity of labeled peptides was analyzed by RP-HPLC on an Agilent 1200 system (Agilent Technologies). A C-18 column (Onyx monolithic, 4.6 mm×100 mm; Phenomenex) was used at a flow rate of 1 mL/min with the following buffer system: buffer A, 0.1% v/v trifluoroacetic acid in H₂O; buffer B, 0.1% trifluoroacetic acid in acetonitrile; with a gradient as follows: 97% buffer A (0-5 min), 97 to 76% buffer A (5-8 min), 76 to 75% buffer A (8-13 min), 75% buffer A (13-25 min). The radioactivity of the eluate was monitored using an in-line NaI radiodetector (Raytest GmbH). Elution profiles were analyzed using GINA STAR™ software (version 2.18; Raytest GmbH).

Octanol/Water Partition Coefficient The radiolabeled peptide (1 MBq) was diluted in 500 μL phosphate-buffered saline (pH 7.4) and mixed vigorously with 500 μL octanol for 2 min using a vortex mixer. The two layers were separated by centrifugation (1000 rpm, 5 min). Samples of 100 μL were taken from each layer and radioactivity was measured with a well-type γ-counter (Wallac Wizard 3″; Perkin-Elmer), and log P values were calculated (n=3).

Cell Culture and Competitive Cell Binding Assay

The GRPR expressing human prostate cancer cell line PC-3 was cultured in Ham's F-12K (Kaighn's) Medium supplemented with 10% fetal calf serum, penicillin (100 units/mL), and streptomycin (100 μg/mL). Cells were grown in tissue culture flasks at 37° C. in a humidified atmosphere containing 5% CO₂. PC-3 xenografts were generated by subcutaneous injection of PC-3 cell suspensions (5×10⁶ cells, 200 μL, 66% RPMI, 33% Matrigel [BD Bioscience]) into male nude BALB/c mice. Xenografts were harvested and snapfrozen to allow cryostat sectioning.

Binding affinities of JMV4168, JMV5132, ^{nat}Ga-JMV4168 and ^{nat}Ga-JMV5132 towards the GRP-receptor were determined in a competitive binding assay on frozen cryostat sections (10-μm thick) of PC-3 xenografts using [¹²⁵I-Tyr⁴]BBN as the competitor radioligand. Tissue sections were pre-incubated for 5 min in ice-cold binding buffer (5 mM MgCl₂, 167 mM Tris-HCl, pH 7.6), followed by incubation for 1 h in binding buffer containing 1% BSA, 5.10⁻¹⁰ M [¹²⁵I-Tyr⁴]BBN and JMV4168, JMV5132, ^{nat}Ga-JMV4168 or ^{nat}Ga-JMV5132 in a range of 10⁻⁶ M to 10⁻¹² M. After incubation, the sections were washed successively with ice-cold binding buffer with 0.25% BSA for 5 min, binding buffer without BSA for 5 min, and ice-cold DI water for 5 seconds. Dried sections were placed in apposition to phosphor screens (PerkinElmer, Super Resolution) for 1 day. Bound Radioactivity was assessed using a phosphor imager system (Cyclone, Packard, model A431201) and quantified using OPTIQUANT™ software. GraphPad Prism software was used to calculate inhibitory concentration of 50% (IC₅₀) values.

Small-Animal PET/CT and Biodistribution Studies

Male nude BALB/c mice (6-8 weeks old) were injected subcutaneously near the right shoulder with a PC-3 cell suspension (5×10⁶ cells, 200 μL, 66% RPMI, 33% MATRIGEL® [BD Bioscience]). 2-3 weeks after inoculation, when tumor size averaged 200 mm³, mice were injected intravenously with 5-10 MBq of radiolabeled peptide (200 pmol, 200 μL). To determine the receptor-mediated localization of the radiolabeled peptides, additional animals were co-injected with an excess (20 nmol) of unlabeled peptide. Mice were euthanized 1 h or 2 h post injection (p.i.) by CO₂/O₂ asphyxiation. Mice were first scanned in prone position on a small animal PET/CT scanner (Inveon; Siemens Preclinical Solutions). PET emission scans were acquired for 30-60 min, followed by a CT scan (spatial resolution 113.15 μm; 80 kV; and 500 μA). Scans were reconstructed using Inveon Acquisition Workplace software (version 1.5; Siemens Preclinical Solutions), using a 3-dimensional ordered-subset expectation maximization/maximization a posteriori algorithm with the following parameters: matrix, 256×256×161; pixel size, 0.40×0.40×0.796 mm; and β-value, 1.5, with uniform variance and FastMAP. After scanning, blood, tumor, and relevant organs and tissues were collected, weighed and counted in a γ-counter. The percentage injected dose per gram (% ID/g) was determined for each tissue sample.

Statistical Analysis

Statistical analysis was performed using GraphPad Prism version 5.01 (San Diego, Calif., USA). IC₅₀ values were determined with datasets from 3 independent experiments, each in duplicate. Data are represented as percentage of total binding (normalized), with SEM and 95% confidence band. An extra sum-of-squares F test was used to compare two best-fit values, and the level of significance was set at P less than 0.05. Biodistribution data are represented as the mean percentage of the injected dose per gram tissue (% ID/g±SD), with group sizes of 3 mice, except at 1 h p.i.: n=2 for Al¹⁸F-JMV5132 and at 2 h p.i.: n=5 for ⁶⁸Ga-JMV4168, Al¹⁸F-JMV5132. Statistical analysis on biodistribution data was performed using a 1-way ANOVA with a Bonferroni post-hoc test, and the level of significance was set at P less than 0.05.

Results

Synthesis of JMV4168 and JMV5132

JMV4168 and JMV5132 (FIG. 1A and FIG. 1B) were synthesized using solid-phase peptide synthesis (Fmoc chemistry). Conjugates were purified by RP-HPLC and characterized by ESI-MS (m/z, [M+2H]²⁺/2: JMV4168, calculated: 815.9414, found: 815.9412; JMV5132, calculated: 821.4416, found: 821.4433). Products were obtained with an average yield of ˜40% and a purity >97% as confirmed by RP-HPLC.

Radiolabeling and Stability Studies

The ⁶⁸Ga-JMV4168 and ⁶⁸Ga-JMV5132 were obtained with a specific activity of 50 MBq/nmol and the Al¹⁸F-JMV5132 with a specific activity of 35 MBq/nmol (88% non-decay corrected yield). RP-HPLC analysis indicated that the radiochemical purity of the Al¹⁸F- or ⁶⁸Ga-labeled peptide preparations used in in-vitro and in-vivo experiments always exceeded 95%. Radio-HPLC elution profiles of ¹⁸F- and ⁶⁸Ga-labeled peptides were determined (not shown). ⁶⁸Ga-JMV4168, ⁶⁸Ga-JMV5132 and Al¹⁸F-JMV5132 had retention times of 14 min, 20 min and 22 min, respectively. Addition of quenchers (methionine, gentisic acid and ascorbic acid) prevented oxidation of the radiolabeled peptides (not shown). Stability studies were performed in reaction mixture and in human serum. Radiolabeled peptides were stable for 2 h in reaction mixture and serum in the presence of quenchers.

Octanol/Water Partition Coefficient

The octanol/water partition coefficients were determined to estimate the lipophilicity of the ¹⁸F- or ⁶⁸Ga-labeled peptides. The log P_{octanol/water} values for ⁶⁸Ga-JMV4168, ⁶⁸Ga-JMV5132 and Al¹⁸F-JMV5132 were −2.53±0.04 and −1.40±0.01 and −1.56±0.08 respectively. The ⁶⁸Ga-DOTA analog (JMV4168) was more hydrophilic than the ⁶⁸Ga- and ¹⁸F-NODA-MPAA analogs (JMV5132).

Competitive Cell Binding Assay

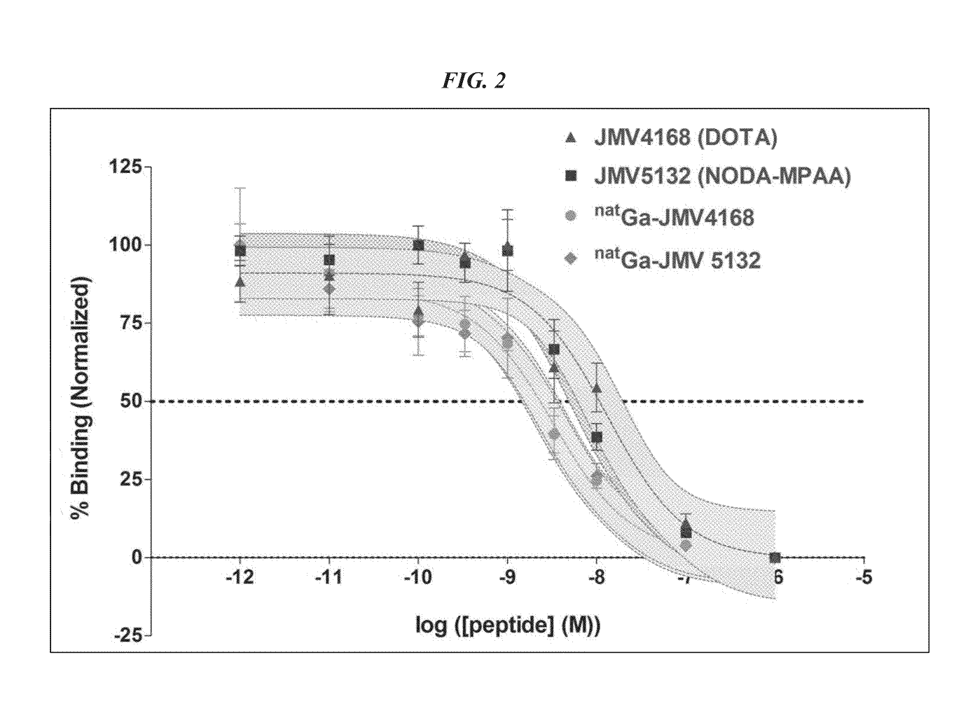

The apparent affinity of JMV5132, JMV4168, ^{nat}Ga-JMV4168 and ^{nat}Ga-JMV5132 for the GRP receptor was determined in a competitive binding assay, using [¹²⁵O-Tyr⁴]BBN as competitor compound/radioligand. The displacement binding curves are shown in FIG. 2. IC₅₀ values for binding to the GRPR [in nM, (95% confidence interval)] for JMV5132 (NODA-MPAA) and JMV4168 (DOTA) were not significantly different: 6.8 nM (4.6-10.0) and 13.2 nM (5.9-29.3)], respectively. IC₅₀ values for ^{nat}Ga-JMV5132 [3.0 (1.5-6.0)] and ^{nat}Ga-JMV4168 [3.2 (1.8-5.9)] were not significantly different, but were significantly lower than those of their unlabeled counterpart, indicating a higher binding affinity for the GRPR.

Small-Animal PET/CT and Biodistribution Studies

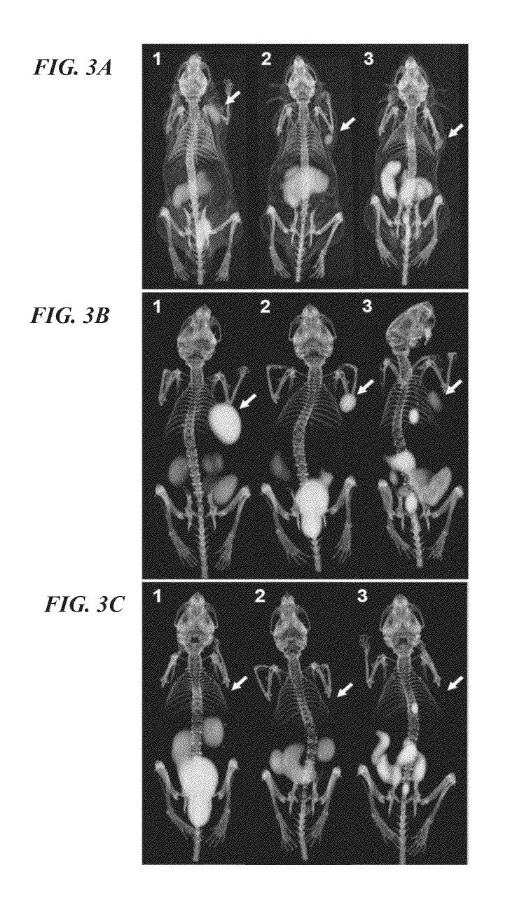

Fused PET and CT images obtained at 1 h and 2 h post-injection (p.i.) are shown in FIG. 3A and FIG. 3B. Maximum intensity projections showed clear visualization of PC-3 tumors with very low background. For all 3 radiolabeled peptides, secretion was predominantly by renal excretion. Partial hepatobiliary excretion was observed for Al¹⁸F-JMV5132 as shown by the nonspecific physiological uptake in the gallbladder and intestines. PET images obtained at 2 h p.i. showed partial clearance of radioactivity in non-target GRPR expressing tissues such pancreas, kidney and intestines as compared to the images obtained at 1 h p.i, which was not the case for tumor.

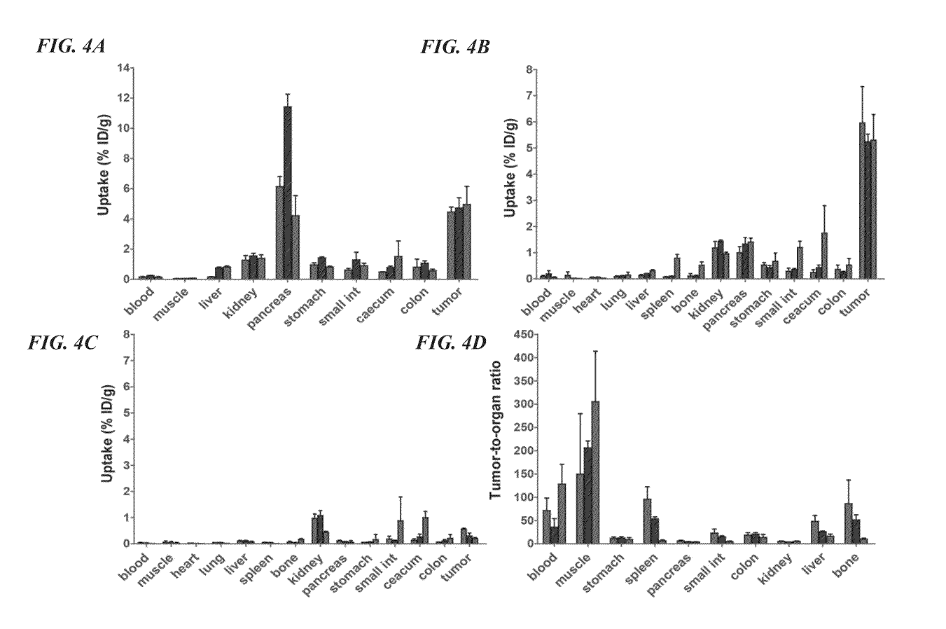

Results of the biodistribution studies of the 3Al¹⁸F- and ⁶⁸Ga-labeled peptides are summarized in FIG. 4A-D. These pharmacokinetic data obtained at 1 h and 2 h p.i. were in line with PET images. High and specific uptake of the tracer was observed in the PC-3 tumors with no significant difference in uptake values between ⁶⁸Ga-JMV4168, ⁶⁸Ga-JMV5132 and Al¹⁸F-JMV5132: 5.96±1.39, 5.24±0.29, 5.30±0.98% ID/g, respectively. Uptake in GRPR-positive organs, such as tumor, pancreas, stomach and intestines was significantly decreased by co-injection with an excess of unlabeled peptide, indicating specific GRPR-targeting. The three tracers displayed fast blood clearance with 0.09±0.04, 0.19±0.13, and 0.05±0.01% ID/g remaining in blood after 2 h p.i. for ⁶⁸Ga-JMV4168, ⁶⁸Ga-JMV5132 and Al¹⁸F-JMV5132, respectively. The three tracers cleared rapidly from the pancreas between 1 h and 2 h p.i., while tumor uptake was preserved. There was an increased uptake of the NOTA-derived tracers Al¹⁸F-JMV5132 and ⁶⁸Ga-JMV5132 in the gallbladder and in the gastro-intestinal tract, with Al¹⁸F-JMV5132 showing the highest uptake values (data not shown). Uptake of the three tracers in other organs like muscle, heart, lung, liver and bone was relatively low (≦0.5% ID/g).

Discussion

The use of radiolabeled GRPR antagonists for targeting tumors in vivo has attracted much attention, starting with somatostatin receptor antagonists showing higher tumor uptake and targeting more receptor binding sites than agonists (Ginj et al., 2006, Proc Natl Acad Sci USA 103:16436-16441). This finding was also extended to GRPR antagonists, with the seminal work of Cescato et al. (Cescato et al., 2008, J Nucl Med 49:318-326). Recently, considerable effort was made to develop radiolabeled GRPR antagonists for imaging GRPR-expressing tumors (Abiraj et al., 2011, J Nucl Med 52:1970-1978; Mansi et al., 2009, Clin Cancer Res 15:5240-5249; Mansi et al., 2011, Eur J Nucl Med Mol imaging 38:97-107; Marsouvanidis et al., 2013, J Med Chem 56:2374-2384; Varasteh et al., 2013, Bioconj Chem 24:1144-1153). These studies revealed favorable pharmacokinetics of radiolabeled antagonists, including high tumor uptake and fast clearance from non-targeted tissues. Several ⁶⁴Cu- and ⁶⁸Ga-labeled receptor antagonists were developed for prostate tumors PET imaging, which showed superior pharmacokinetics compared to ⁶⁴Cu or ¹⁸F-labeled GRPR agonists described in the literature (Abiraj et al., 2011, J Nucl Med 52:1970-1978; Mansi et al., 2009, Clin Cancer Res 15:5240-5249; Mansi et al., 2011, Eur J Nucl Med Mol imaging 38:97-107).

We report here on the development of an NODA-MPAA-conjugated GRPR-antagonist (JMV5132) labeled with Al¹⁸F for PET-imaging of GRPR-positive tumors and the direct comparison with ⁶⁸Ga-radiolabeled analogs. In our previous work, the statin-based GRPR antagonist JMV594 was linked to DOTA via a (βAla)₂ linker and labeled with ¹¹¹In. It showed very good tumor targeting in PC-3 xenograft mice (Marsouvanidis et al., 2013, J Med Chem 56:2374-2384). In the present study, we conjugated JMV594 to NODA-MPAA, using the same linker, for radiolabeling with Al¹⁸F to obtain/designated JMV5132. We labeled JMV5132 with both Al—F and Ga and compared these tracers with the DOTA-(βAla)₂-JMV594 peptide (JMV4168) labeled with ⁶⁸Ga.

The radiolabeling of peptides via complexation of Al¹⁸F by a NOTA chelator was first described by McBride et al. (McBride et al., 2009, J Nucl Med 50:991-998). This novel technique was successfully applied to several peptides, including a GRPR agonist (Dijkgraaf et al., 2012, J Nucl Med 53:947-952) and recently a GRPR antagonist (Liu et al., Nov. 6, 2013, J Nucl Med [Epub ahead of print]). Recently, McBride et al. reported the labeling of peptides with Al¹⁸F in a one-pot one-step procedure using the NODA-MPAA chelator (D'Souza et al., 2011, Bioconj Chem 22:1793-1803; McBride et al. 2012, Bioconj Chem 23:538-547), leading to a kit formulation, after which the labeled peptide could be purified by solid-phase extraction (SPE).

In the present study, we optimized the labeling conditions further to achieve Al¹⁸F-labeled JMV5132 in less than 20 min with complete incorporation of ¹⁸F-fluoride, resulting in a high specific activity (35 MBq/nmol), without the need for SPE further purification. Recently, radiolabeling of the NODA-GA-derived GRPR-antagonist RM1 with Al¹⁸F was described (Liu et al., Nov. 6, 2013, J Nucl Med [Epub ahead of print]), but resulted in a very low radiochemical yield (5% decay-corrected) and low specific activity (1.85 MBq/nmol), which was likely caused by the chelator used, i.e. NODA-GA.

In receptor binding studies using PC-3 tumor sections, the in vitro affinities of JMV5132 and JMV4168 were comparable, as shown by the similar IC₅₀ values, indicating that the type of chelator did not affect the affinity. The peptides labeled with ^{nat}Ga had slightly higher receptor affinities than their unlabeled counterpart as shown by the lower IC₅₀ values, indicating that the presence of Ga³⁺ in the chelator enhanced the affinity of the peptides for the GRP receptor, most likely by inducing structural changes.

The PET images obtained with Al¹⁸F-JMV5132 showed higher spatial resolution as compared to the images obtained with the ⁶⁸Ga-labeled tracers, which is most likely due to the longer positron range of ⁶⁸Ga (Disselhorst et al., 2010, J Nucl Med 51:610-617).

The comparative biodistribution study showed GRPR-specific accumulation of all radiolabeled GRPR antagonists in the tumor. Al¹⁸F-JMV5132, ⁶⁸Ga-JMV5132 and ⁶⁸Ga-JMV4168 tracers showed similar uptake in the GRPR-positive organs, such as PC-3 tumor, pancreas, stomach and colon. The uptake was receptor-mediated as shown by the reduction of the tracer uptake in tumor and other receptor-positive organs after co-injection of excess unlabeled peptide. However, the wash-out from receptor-positive organs occurred at different rates, with higher retention of the tracers in the tumor than in the pancreas. The tumor uptake was retained while pancreas uptake decreased by a factor of 6.1, 8.6 and 3.0 from 1 h to 2 h p.i., for ⁶⁸Ga-JMV4168, ⁶⁸Ga-JMV5132 and Al¹⁸F-JMV5132, respectively.

Despite its low internalization rate, the high tumor uptake and persistent tumor retention of these antagonist tracers was expected, as it was previously described for a few other radiolabeled antagonists (Cescato et al., 2008, J Nucl Med 49:318-326; Mansi et al., 2009, Clin Cancer Res 15:5240-5249; Mansi et al., 2011, Eur J Nucl Med Mol imaging 38:97-107; Varasteh et al., 2013, Bioconj Chem 24:1144-1153). The reason for the higher retention of antagonists in tumor tissue may be a higher number of binding sites for the antagonists compared to the agonists, a higher metabolic stability of antagonists, or a very strong interaction of the antagonist with the receptor (Cescato et al., 2008, J Nucl Med 49:318-326; Mansi et al., 2011, Eur J Nucl Med Mol imaging 38:97-107). Moreover, previous studies using radiolabeled GRPR-antagonists, also reported a faster clearance from the pancreas (and abdominal organs) was observed between 1 h p.i. and 4 h p.i. as compared to tumors. These data were in contrast to data of radiolabeled GRPR agonists showing retention of activity in the abdominal region for a longer period of time. A few reasons for these differences in tissue clearance kinetics between tumor and pancreas have been postulated, such as species differences, or more efficient perfusion of the pancreas and intestine (Mansi et al., 2011, Eur J Nucl Med Mol imaging 38:97-107). Another explantation for the faster washout from the pancreas may be a potential difference in metabolic degradation of the peptide by enzymes in the pancreas.

Clearance from background tissues such as blood, muscle, heart, lung, liver and bone was very fast for all 3 radioligands. This led to very high tumor-to-background ratios with all tracers allowing for clear visualization of the tumor. Overall, Al¹⁸F-JMV5132 showed improved imaging properties compared to the previously reported Al¹⁸F-NOTA-8-Aoc-BBN(7-14)NH₂ GRPR agonist (Dijkgraaf et al., 2012, J Nucl Med 53:947-952), showing lower tumor uptake, much higher pancreatic uptake, and higher liver and intestinal uptake in the same animal model.

The increased uptake of Al¹⁸F-JMV5132 and ⁶⁸Ga-JMV5132 in the gallbladder and gastro-intestinal excretions may indicate partial hepatobiliary excretion of the tracers due to their higher lipophilicity, which may be partially caused by the benzyl group. Considering the clinical application of the tracers, the higher signal intensity in the intestines using this tracer may affect visualization of prostate-confined tumor or spread to lymph nodes. Nevertheless, considering the superior imaging characteristics of ¹⁸F, further development of Al¹⁸F-JMV5132 as a tracer for PC diagnostic and therapy follow-up is warranted.

### CONCLUSION

High sensitivity and receptor-specific imaging of PC with PET/CT can be achieved using ⁶⁸Ga- and Al¹⁸F-labeled GRPR-antagonists. In this study, labeling of JMV5132 with Al¹⁸F could be performed within 20 min with high specific activity without the need for purification. Despite superior PET imaging characteristics of Al¹⁸F-JMV5132 with higher resolution, the ⁶⁸Ga-JMV4168 tracer showed the most favorable biodistribution with low hepatobiliary excretion. These new PET tracers will allow imaging, detection and diagnosis of prostate cancer and other GRPR-expressing tumors.

### Example 2

### Synthesis and Labeling of IMP468 Bombesin Peptide

The ¹⁸F labeled targeting moieties can include any molecule that binds specifically or selectively to a cellular target that is associated with or diagnostic of a disease state or other condition that may be imaged by ¹⁸F PET. Bombesin is a 14 amino acid peptide that is homologous to neuromedin B and gastrin releasing peptide, as well as a tumor marker for cancers such as lung and gastric cancer and neuroblastoma. IMP468 (NOTA-NH—(CH₂)₇CO-Gln-Trp-Val-Trp-Ala-Val-Gly-His-Leu-Met-NH₂; SEQ ID NO:3) was synthesized as a bombesin analogue and labeled with ¹⁸F to target the gastrin-releasing peptide receptor.

The peptide was synthesized by Fmoc based solid phase peptide synthesis on Sieber amide resin, using a variation of a synthetic scheme reported in the literature (Prasanphanich et al., 2007, PNAS USA 104:12463-467). The synthesis was different in that a bis-t-butyl NOTA ligand was add to the peptide during peptide synthesis on the resin.

IMP468 (0.0139 g, 1.02×10⁻⁵ mol) was dissolved in 203 μL of 0.5 M pH 4.13 NaOAc buffer. The peptide dissolved but formed a gel on standing so the peptide gel was diluted with 609 μL of 0.5 M pH 4.13 NaOAc buffer and 406 μL of ethanol to produce an 8.35×10⁻³ M solution of the peptide. The ¹⁸F was purified on a QMA cartridge and eluted with 0.4 M KHCO₃ in 200 μL fractions, neutralized with 10 μL of glacial acetic acid. The purified ¹⁸F, 40 μL, 1.13 mCi was mixed with 3 μL of 2 mM AlCl₃ in pH 4, 0.1 M NaOAc buffer. IMP468 (59.2 μL, 4.94×10⁻⁷ mol) was added to the Al¹⁸F solution and placed in a 108° C. heating block for 15 min. The crude product was purified on an HLB column, eluted with 2×200 μL of 1:1 EtOH/H₂O to obtain the purified ¹⁸F-labeled peptide in 34% yield.

### Example 3

### Imaging of Tumors Using ¹⁸F Labeled Bombesin

A NOTA-conjugated bombesin derivative (IMP468) was prepared as described above. We began testing its ability to block radiolabeled bombesin from binding to PC-3 cells as was done by Prasanphanich et al. (PNAS 104:12462-12467, 2007). Our initial experiment was to determine if IMP468 could specifically block bombesin from binding to PC-3 cells. We used IMP333 as a non-specific control. In this experiment, 3×10⁶ PC-3 cells were exposed to a constant amount (˜50,000 cpms) of ¹²⁵I-Bombesin (Perkin-Elmer) to which increasing amounts of either IMP468 or IMP333 was added. A range of 56 to 0.44 nM was used as our inhibitory concentrations.

The results showed that we could block the binding of ¹²⁵I-BBN with IMP468 but not with the control peptide (IMP333) (not shown), thus demonstrating the specificity of IMP468. Prasanphanich indicated an IC₅₀ for their peptide at 3.2 nM, which is approximately 7-fold lower than what we found with IMP468 (21.5 nM).

This experiment was repeated using a commercially available BBN peptide. We increased the amount of inhibitory peptide from 250 to 2 nM to block the ¹²⁵I-BBN from binding to PC-3 cells. We observed very similar IC₅₀-values for IMP468 and the BBN positive control with an IC₅₀-value higher (35.9 nM) than what was reported previously (3.2 nM) but close to what the BBN control achieved (24.4 nM).

To examine in vivo targeting, the distribution of Al¹⁸F(IMP468) was examined in scPC3 prostate cancer xenograft bearing nude male mice; alone vs. blocked with bombesin. For radiolabeling, aluminum chloride (10 μL, 2 mM), 51.9 mCi of ¹⁸F (from QMA cartridge), acetic acid, and 60 μL of IMP468 (8.45 mM in ethanol/NaOAc) were heated at 100° C. for 15 min. The reaction mixture was purified on reverse phase HPLC. Fractions 40 and 41 (3.56, 1.91 mCi) were pooled and applied to HLB column for solvent exchange. The product was eluted in 800 μL (3.98 mCi) and 910 μCi remained on the column. iTLC developed in saturated NaCl showed 0.1% unbound activity.

A group of six tumor-bearing mice were injected with Al¹⁸F(IMP468) (167 μCi, ˜9×10⁻¹⁰ mol) and necropsied 1.5 h later. Another group of six mice were injected iv with 100 μg (6.2×10⁻⁸ mol) of bombesin 18 min before administering Al¹⁸F(IMP468). The second group was also necropsied 1.5 h post injection. The data showed specific targeting of the tumor with [Al¹⁸F] IMP 468 (not shown). Tumor uptake of the peptide was reduced when bombesin was given 18 min before the Al¹⁸F(IMP468) (not shown). Biodistribution data indicates in vivo stability of Al¹⁸F(IMP468) for at least 1.5 h (not shown).

Larger tumors showed higher uptake of Al¹⁸F(IMP468), possibly due to higher receptor expression in larger tumors (not shown). The biodistribution data showed Al¹⁸F(IMP468) tumor targeting that was in the same range as reported for the same peptide labeled with ⁶⁸Ga by Prasanphanich et al. (not shown). The results demonstrate that the ¹⁸F peptide labeling method can be used in vivo to target receptors that are upregulated in tumors, using targeting molecules besides antibodies. In this case, the IMP468 targeting took advantage of a naturally occurring ligand-receptor interaction. The tumor targeting was significant with a P value of P=0.0013. Many such ligand-receptor pairs are known and any such targeting interaction may form the basis for ¹⁸F imaging, using the methods described herein.

### Example 4

### Preparation of Al¹⁹F Peptides

An improved method for preparing [Al¹⁹F] compounds was developed. IMP461 was prepared and labeled with ¹⁹F. The peptide was synthesized on Sieber amide resin with the amino acids and other agents added in the following order Aloc-D-Lys(Fmoc)-OH, Trt-HSG-OH, Aloc removal, Fmoc-D-Tyr(But)-OH, Aloc-D-Lys(Fmoc)-OH, Trt-HSG-OH, Aloc removal, Fmoc-D-Ala-OH, and Bis-t-butylNOTA. The peptide was then cleaved and purified by HPLC to afford the product IMP461 ESMS MH⁺ 1294 NOTA-D-Ala-D-Lys(HSG)-D-Tyr-D-Lys(HSG)-NH₂; SEQ ID NO:4). Reacting IMP461 with AlCl₃+NaF resulted in the formation of three products (not shown). However, by reacting IMP461 with AlF₃.3H₂O we obtained a higher yield of Al¹⁹F(IMP461).

Synthesis of IMP 473: [Al¹⁹F(IMP461)] To (14.1 mg, 10.90 μmol) IMP461 in 2 mL NaOAc (2 mM, pH 4.18) solution added (4.51 mg, 32.68 μmol) AlF₃.3H₂O and 500 μL ethanol. The pH of the solution to adjusted to 4.46 using 3 μL 1 N NaOH and heated in a boiling water bath for 30 minutes. The crude reaction mixture was purified by preparative RP-HPLC to yield 4.8 mg (32.9%) of IMP 473. HRMS (ESI-TOF) MH expected 1337.6341; found 1337.6332

These results demonstrate that ¹⁹F labeled molecules may be prepared by forming metal-¹⁹F complexes and binding the metal-¹⁹F to a chelating moiety, as discussed above for ¹⁸F labeling. The instant Example shows that a targeting peptide of use for MRI imaging may be prepared using the instant methods.

What is claimed is:

1. A method of detecting or imaging prostate cancer comprising:
a) administering to a subject with prostate cancer a bombesin analog labeled with Al¹⁸F or ⁶⁸Ga; and
b) detecting the distribution of labeled bombesin analog by positron emission tomography (PET) or SPECT to image the prostate cancer.

2. The method of claim 1, wherein the bombesin analog is a GRPR antagonist.

3. The method of claim 2, wherein the GRPR antagonist is JMV5132 [NODA-MPAA-(βAla)₂-H-d-Phe-Gln-Trp-Ala-Val-Gly-His-Sta-Leu-NH₂] (SEQ ID NO: 5) or JMV4168 [DOTA-(βAla)₂-H-d-Phe-Gln-Trp-Ala-Val-Gly-His-Sta-Leu-NH₂] (SEQ ID NO: 6).

4. The method of claim 1, further comprising analyzing the distribution of Al¹⁸F-labeled or or ⁶⁸Ga-labeled molecule to detect prostate cancer in the subject.

5. The method of claim 1, further comprising analyzing the distribution of Al¹⁸F-labeled or ⁶⁸Ga-labeled molecule to detect or diagnose prostate cancer in the subject.

6. The method of claim 1, further comprising detecting metastatic prostate cancer.

7. A method of imaging prostate cancer comprising:
a) administering to a subject with prostate cancer an Al¹⁹F-labeled bombesin analog that binds to prostate cancer cells; and
b) detecting the distribution of labeled bombesin analog by magnetic resonance imaging (MRI) to image the prostate cancer.

8. The method of claim 7, wherein the bombesin analog is a GRPR antagonist.

9. The method of claim 8, wherein the wherein the GRPR antagonist is JMV5132 [NODA-MPAA-(βAla)₂-H-d-Phe-Gln-Trp-Ala-Val-Gly-His-Sta-Leu-NH₂] (SEQ ID NO: 5) or JMV4168 [DOTA-(βAla)₂-H-d-Phe-Gln-Trp-Ala-Val-Gly-His-Sta-Leu-NH₂] (SEQ ID NO: 6).

10. The method of claim 7, further comprising analyzing the distribution of Al¹⁹F-labeled molecule to detect or diagnose rostate cancer in the subject.

11. The method of claim 7, further comprising detecting metastatic prostate cancer.

12. A composition comprising JMV5132 [NODA-MPAA-(βAla)₂-H-d-Phe-Gln-Trp-Ala-Val-Gly-His-Sta-Leu-NH₂] (SEQ ID NO: 5) or JMV4168 [DOTA-(βAla)₂-H-d-Phe-Gln-Trp-Ala-Val-Gly-His-Sta-Leu-NH₂] (SEQ ID NO: 6).

13. The composition of claim 12, further comprising metal-¹⁸F, metal-¹⁹F or ⁶⁸Ga attached to the NODA or DOTA.

14. The composition of claim 13, wherein the metal is selected from the group consisting of aluminum, gallium, indium, and thallium.

15. The composition of claim 13, wherein the metal is aluminum.

16. The composition of claim 12, further comprising at least one component selected from the group consisting of water, aluminum, trehalose, potassium biphthalate, ethanol, and ascorbic acid.

17. The composition of claim 12, wherein the composition has a pH between 3.9 and 4.2.
